(19) United States
(12) Patent Application Publication
(10) Pub. No.: US 2022/0323963 A1
(43) Pub. Date: Oct. 13, 2022

(54) SAMPLE PROCESSING CARTRIDGE FOR USE WITH A DNA SEQUENCER

(71) Applicant: The Johns Hopkins University, Baltimore, MD (US)

(72) Inventors: Korine A. Ohiri, Laurel, MD (US); Andrea C. Timm, Ellicott City, MD (US); Peter M. Thielen, Catonsville, MD (US); Tobias D. Dilworth, Fulton, MD (US); Hayley M. DeHart, Sykesville, MD (US); Tessa B. VanVolkenburg, Ellicott City, MD (US)

(21) Appl. No.: 17/552,181

(22) Filed: Dec. 15, 2021

Related U.S. Application Data
(60) Provisional application No. 63/172,842, filed on Apr. 9, 2021.

(51) Int. Cl. B01L 7/00; B01L 3/00; G01N 35/10; C12Q 1/686

(52) U.S. Cl. CPC B01L 7/52; B01L 3/50851; G01N 35/1095; C12Q 1/686; B01L 2200/0673; G01N 2035/00465

(57) ABSTRACT

A multi-module sample preparation device for use with a DNA sequencer is provided. The device includes several modules that are operatively connected in a manner such that a liquid sample containing DNA for analysis can be charged into the device and automatically prepared for sequencing with little or no user interaction. The device enables targeted amplification, purification, and library preparation for a liquid sample prior to being injected into a DNA sequencer.

### CROSS-REFERENCE TO RELATED APPLICATIONS

This application claims the benefit of U.S. Provisional Application No. 63/172,842 filed on Apr. 9, 2021, the entire contents of which are hereby incorporated herein by reference

### TECHNICAL FIELD

Example embodiments relate generally to sample processing devices that prepare a biological sample (e.g., a biological sample suspended in a liquid matrix) for DNA sequencing, in which the biological sample may be prepared for DNA sequencing with minimal or no interaction by a user.

### BACKGROUND

Commercial sample preparation devices currently have a rather large footprint, multiple independent hardware components, and depend on a high degree of user interaction. Additionally, such devices are not designed to work outside of a laboratory environment.

### SUMMARY OF THE DISCLOSURE

Certain embodiments disclosed herein provide a multi-module sample preparation device (e.g., cartridge or chip) that includes a sample inlet for receiving a liquid sample comprising DNA, a sample outlet (e.g., to be delivered to a DNA sequencer), a waste outlet, and a plurality of operatively connected modules. The multi-module sample preparation device includes a first lyophilized chamber module comprising a plurality of lyophilized PCR primers and a lyophilized PCR master mix including one or more deoxynucleoside triphosphates (dNTPs), one or more buffers, and/or one or more polymerases. The first lyophilized chamber module may include a first lyophilized chamber inlet operatively connected to the sample inlet, and a first lyophilized chamber outlet. The multi-module sample preparation device may also include a first mixing module comprising a first mixing module inlet operatively connected to the first lyophilized chamber outlet, and a first mixing module outlet. The multi-module sample preparation device may also include an amplification module, such as a PCR module comprising a serpentine microfluidic channel and a plurality of discrete heaters in operative communication with a plurality of predetermined zones of the serpentine microfluidic channel oriented to produce one or more amplified target DNA regions. The PCR module, for example, may include a PCR inlet operatively connected to the first mixing module outlet, and a PCR outlet. The multi-module sample preparation device may also include a purification module comprising an active region including a solid phase configured to bind and release the one or more amplified target DNA regions, in which the purification module includes a purification inlet in operative communication with the PCR outlet, and a purification outlet being operatively and selectively connected with a first pathway from the purification outlet to the waste outlet and a second pathway from the purification outlet to a purified stream outlet. The multi-module sample preparation device may also include a second lyophilized chamber module comprising a plurality of lyophilized adapter sequences for enabling sequencing of the amplified target DNA regions, in which the second lyophilized chamber module includes a second lyophilized chamber module inlet operatively connected to the purified stream outlet, and a second lyophilized chamber module outlet. The multi-module sample preparation device may also include a third lyophilized chamber module comprising a lyophilized sequencing loading buffer composition, in which the third lyophilized chamber module includes a third lyophilized chamber module inlet operatively connected to a source of a reconstitution fluid, and a third lyophilized chamber module outlet. The multi-module sample preparation device may also include a second mixing module comprising one or more second mixing pools, in which the second mixing module includes one or more second mixing module inlets operatively connected to the second lyophilized chamber module outlet and the third lyophilized chamber module outlet, and a second mixing module outlet connected to the sample outlet.

In another aspect, the invention provides a system optionally including a liquid sample collection apparatus including a collection apparatus outlet and a multi-module sample preparation device, such as those described and disclosed herein, in which the sample inlet of the multi-module sample preparation device is in operative communication with the collection apparatus outlet. The system may also comprise a DNA sequencer, in which the DNA sequencer is in operative communication with the sample outlet of the multi-module sample preparation device. In certain example embodiments, the system may include a sequencer interface module located between and in operative communication with the sample outlet of the multi-module sample preparation device and the DNA sequencer.

In yet another aspect, the present invention provides a method of preparing a sample for DNA sequencing, in which the method may include the following: (a) optionally collecting a liquid sample, (b) feeding a liquid sample (e.g., lysed sample) into a multi-module sample preparation device, such as those described and disclosed herein; (c) flowing the liquid sample through a first lyophilized chamber module and reconstituting the plurality of lyophilized PCR primers and the lyophilized PCR mastermix; (d) flowing the liquid sample from the first lyophilized chamber into the first mixing module forming a homogenous PCR-ready liquid sample; (e) flowing the PCR-ready liquid sample from the first mixing module into and through the PCR module, and performing an amplification process within the PCR module and forming an amplified liquid sample; (f) flowing the amplified liquid sample from the PCR module into and through the purification module forming a purified liquid sample; (g) flowing the purified liquid sample into and through the second lyophilized chamber module and reconstituting the plurality of lyophilized adapter sequences and allowing attachment of the adapter sequences to the one or more amplified target DNA regions forming a sequence-able DNA sample; (h) flowing a reconstitution fluid into and through the third lyophilized chamber module and reconstituting the lyophilized sequencing loading buffer composition forming a liquid sequencing buffer solution; and (i) flowing the sequence-able DNA liquid sample into the second mixing module and flowing the liquid sequencing buffer solution into the second mixing module, and mixing the sequence-able DNA liquid sample and the liquid sequencing buffer solution forming a sequencing-ready liquid sample.

### BRIEF DESCRIPTION OF THE DRAWING(S)

Example embodiments will now be described more fully hereinafter with reference to the accompanying drawings, in which some, but not all embodiments are shown. Indeed, the technology described herein may be embodied in many different forms and should not be construed as limited to the embodiments set forth herein; rather, these embodiments are provided so that this disclosure will satisfy applicable legal requirements. Like numbers refer to like elements throughout, and wherein:

FIG. 1 illustrates a schematic of a multi-module sample preparation device according to certain example embodiments;

FIG. 2 illustrates a schematic of a DNA sequencer flow cell (e.g., a MinION flow cell);

FIG. 3 illustrates a sequencer interface in an assembled configuration according to certain example embodiments;

FIG. 4 illustrates an exploded view of a sequencer interface including the multi-port rotary valve according to certain example embodiments;

FIG. 5 illustrates an alternative exploded view of a sequencer interface including the multi-port rotary valve according to certain example embodiments;

FIG. 6 illustrates a schematic of a manifold component of a sequencer interface according to certain example embodiments;

FIG. 7 illustrates a transparent schematic of the sequencer interface of FIG. 6 and illustrates the respective orientation of the ports associated with a manifold component of the sequencer interface in accordance with certain example embodiments;

FIG. 8 illustrates a transparent schematic of the multi-port rotary valve in accordance with certain example embodiments;

FIG. 9 illustrates a non-transparent schematic of the multi-port rotary valve of FIG. 8 in accordance with certain example embodiments;

FIG. 10A illustrates the multi-port rotary valve in a first position (e.g., priming of the DNA sequencer with priming buffer solution while a sequencing-ready liquid sample fills an injection loop) in accordance with certain example embodiments;

FIG. 10B illustrates the multi-port rotary valve in a second position (e.g., priming buffer solution pushes the sequencing-ready liquid sample out of the injection loop and into the DNA sequencer) in accordance with certain example embodiments;

FIG. 11 illustrates a general schematic for a system in accordance with certain example embodiments; and

FIG. 12 illustrates a flow chart for a method in accordance with certain example embodiments.

### DETAILED DESCRIPTION

Some example embodiments will be described more fully hereinafter with reference to the accompanying drawings, in which some, but not all example embodiments are shown. Indeed, the examples described and pictured herein should not be construed as being limited to the scope, applicability, or configuration of the present disclosure. Rather, these example embodiments are provided so that this disclosure will satisfy applicable legal requirements. As used in the specification, and in the appended claims, the singular forms “a”, “an”, “the”, include plural referents unless the context clearly dictates otherwise. Like reference numerals refer to like elements throughout.

Example embodiments herein relate generally to sequencing preparation for a variety of liquid samples, such as environmental DNA (eDNA) from water samples as only one example. A liquid sample for preparation for DNA sequencing, such as from an auto-sampler that may perform lysis of the cells in the sample to provide fresh lysate for being prepared for DNA sequencing. In this regard, lysate for DNA sequencing may generally require the amplification, such as by polymerase chain reaction (PCR) techniques, purification of the amplified target DNA regions, and library preparation prior to injection into a DNA sequencer. In accordance with certain example embodiments, a multi-module sample preparation device (e.g., embodied as a cartridge or a chip including the modules) may contain a plurality of interconnected but independent modules to accomplish each of the steps necessary for automating sample preparation for DNA sequencing with little or no user interaction. As different steps and/or modules within the multi-module sample preparation device may require a variety of reagents for accomplishing a particular step, such reagents may be preloaded in the appropriate module. Accordingly, the multi-module sample preparation device may enable a “hands-off” sample preparation process, which effectively eliminates user error and enables in-field or on-site sample preparation for DNA sequencing. In this regard, the multi-module sample preparation device may be beneficially employed in both laboratory environments and point-of-sample acquisition (e.g., in a field setting and/or underwater environments, such as submersible autonomous underwater vehicle (AUV). Individual modules, as discussed in more detail below, may be fabricated on a chip, such as described and disclosed herein, and in the case of the purification module and lyophilization modules, either packed, functionalized, or filled with the appropriate reagents before shelf-stabilization and sealing.

In example embodiments, a liquid sample including one or more lysates may be delivered to the multi-module sample preparation device from, by way of example only, an upstream sample collector. The liquid sample will reconstitute lyophilized primers and polymerase chain reaction (PCR) master mix in a first module, such as a first lyophilized chamber module, and then mix in a second module, such as a first mixing module. After reconstitution and mixing of the lyophilized primers and PCR master mix within the liquid sample, the resulting solution will then flow into an amplification module, such as a PCR module. In certain example embodiments, the PCR module may perform a fixed number of PCR cycles by passing the liquid sample through different temperature zones generated by heaters adhered (or alternatively operatively connected) to the multi-module sample preparation device, or alternatively a common “bus” or jig that this multi-module sample preparation device sits into and interfaces therewith. By way of example, the bus may include integrated heaters and allow for all of the necessary fluidic connections. After amplification, the now amplified liquid sample will flow into the purification module that removes at least a portion of undesired constituents (e.g., residual primer sequences, dNTPs, and salts). The purification module, for example, may perform a cleanup and purification of the amplified liquid sample using solid phase extraction (SPE) techniques. A waste outlet, in accordance with certain example embodiments, may be incorporated into the end of this module and allow for removal of excess PCR product and the reagents required for SPE. Following rinsing and prior to elution, for example, the exit port (e.g., waste outlet) from this module may be switched to connect to an inlet of the subsequent module, such as by using a simple valve. The purified liquid sample may then be passed to a second lyophilized chamber module filled with lyophilized sequencing adapters, for example, a rapid adapter from Oxford Nanpore Technologies, for sequencing preparation. While the purified liquid sample is incubating in a delay circuit of the second lyophilized chamber module, such as for about 5 minutes (which may be altered depending the desired incubation time), a third lyophilized chamber module including, for example, a lyophilized sequencing loading buffer and optionally loading solution may be reconstituted. Accordingly, two separate reagent pools (e.g., one in the second lyophilized chamber module and a second in the third lyophilized chamber module) may be mixed in a second mixing module to form a purified and sequencer-ready liquid sample, which may be transported off the multi-module sample preparation device (e.g., sample preparation chip) to a sequencer interface, such as multi-port rotary valve, for injection into a DNA sequencer.

As referenced above, the multi-module sample preparation device may be embodied as a single-use cartridge or chip housing the plurality of modules utilized in the sample preparation for DNA sequencing. For example, the multi-module sample preparation device may comprise a micro-fluidic chip in which each of the modules (e.g., micro-channels and/or chambers) may, by way of example only, have been 3D printed, or hot embossed, or etched and/or molded into a material (glass, silicon, or a plastic such as polydimethylsiloxane—PDMS or polymethyl methacrylate—PMMA, a polycarbonate, or pre-fluorinated polymers). In this regard, the micro-channels and/or chambers forming each of the modules of the micro-fluidic chip are connected together in order to achieve the desired features (e.g., reconstitution of lyophilized reagents, mixing, controlling residence time for a given portion of a given module, etc.). The micro-channels and/or chambers forming each of the modules of the micro-fluidic chip may have different inner diameters, for example, ranging from 5 to 2500 microns, such as at least about any of the following: 5, 10, 25, 50, 80, 100, 120, 150, 180, and 200 microns, and/or at most about any of the following: 2500, 2000, 1500, 1000, 800, 600, 500, 450, 400, 350, 300, 250, and 200 microns. The network of micro-channels and/or chambers formed into the micro-fluidic chip may be connected to the outside by inputs and outputs pierced through the chip or through the use of a flangeless connector interfaced to the network of micro-channels and/or chambers via one or more tapped ports or by the use of face compression and adhesive seals. It is through these holes that the liquids (or gases) may be injected and removed from the micro-fluidic chip (e.g., through tubing, syringe adapters or even simple holes in the chip) with external active systems (e.g., pressure controller, syringe-pump or peristaltic pump) or passive ways (e.g. hydrostatic pressure).

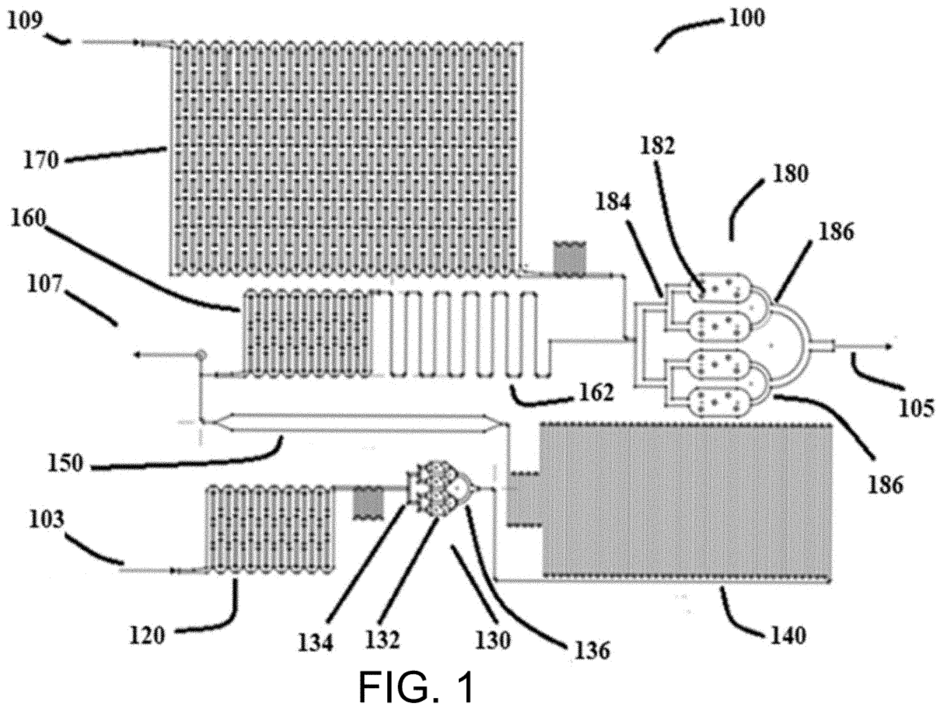

As illustrated in FIG. 1, certain example embodiments provide a multi-module sample preparation device 100 (e.g., cartridge or micro-fluidic chip) that includes a sample inlet 103 for receiving a liquid sample comprising DNA, a sample outlet 105 (e.g., to be delivered to a DNA sequencer), a waste outlet 107, and a plurality of operatively connected modules. The multi-module sample preparation device 100 includes a first lyophilized chamber module 120 comprising a plurality of lyophilized PCR primers and a lyophilized PCR master mix including one or more deoxynucleoside triphosphates (dNTPs), one or more buffers, and/or one or more polymerases. The first lyophilized chamber module 120 may include a first lyophilized chamber inlet operatively connected to the sample inlet 103, and a first lyophilized chamber outlet. The multi-module sample preparation device 100 may also include a first mixing module 130 comprising one or more first mixing pools 132 (or alternatively a serpentine, non-pooling mixer, a static mixer, or a plurality of micro-fluidic channels), in which the first mixing module includes a first mixing module inlet operatively connected to the first lyophilized chamber outlet, and a first mixing module outlet. The multi-module sample preparation device 100 may also include a PCR module 140 comprising a serpentine microfluidic channel and a plurality of discrete heaters in operative communication with a plurality of predetermined zones of the serpentine microfluidic channel oriented to produce one or more amplified target DNA regions. The PCR module 140, for example, may include a PCR inlet operatively connected to the first mixing module outlet, and a PCR outlet. The multi-module sample preparation device 100 may also include a purification module 150 comprising an active region including a solid phase (e.g., packed bed and/or functionalized surfaces of the module) configured to bind and release the one or more amplified target DNA regions, in which the purification module 150 includes a purification inlet in operative communication with the PCR outlet, and a purification outlet being operatively and selectively connected with a first pathway from the purification outlet to the waste outlet 107 and a second pathway from the purification outlet to a purified stream outlet. The multi-module sample preparation device 100 may also include a second lyophilized chamber module 160 comprising a plurality of lyophilized adapter sequences for enabling sequencing of the amplified target DNA regions, in which the second lyophilized chamber module 160 includes a second lyophilized chamber module inlet operatively connected to the purified stream outlet, and a second lyophilized chamber module outlet. The multi-module sample preparation device 100 may also include a third lyophilized chamber module 170 comprising a lyophilized sequencing buffer composition, in which the third lyophilized chamber module 170 includes a third lyophilized chamber module inlet operatively connected to a source of a reconstitution fluid 109, and a third lyophilized chamber module outlet. The multi-module sample preparation device 100 may also include a second mixing module 180 comprising one or more second mixing pools 182, in which the second mixing module 180 includes one or more second mixing module inlets operatively connected to the second lyophilized chamber module outlet and the third lyophilized chamber module outlet, and a second mixing module outlet connected to the sample outlet 105.

In certain example embodiments, the first step in sample preparation requires combining the PCR primers and master mix with the lysate of the liquid sample. In this regard, the first lyophilized chamber module 120 includes a preloaded and lyophilized mixture of the PCR primers and master mix. For example, the PCR primers and master mix have been lyophilized and stored in the first lyophilized chamber module 120 prior to use. The liquid sample, for example, may enter into the first lyophilized chamber module 120 and reconstitute the lyophilized mixture of the PCR primers and master mix. In certain example embodiments, the first lyophilized chamber module 120 may comprise a capillary bed configuration that exerts a capillary force on the liquid sample that is greater than gravitational forces acting on the liquid sample. For example, the capillary bed configuration may include a plurality of posts or columns that may extend at least a portion (or completely) of the distance of the bed depth, in which the plurality of posts or columns may be provided in a high density. In this regard, the bed depth and the distance between the posts or columns in both the X and Y directions impact the filling properties and strength of the capillary force. In accordance with certain example embodiments, the capillary bed configuration may be formed with at least the following three (3) design criteria: (1) hold the correct volume; (2) maximize capillary force; and (3) fit within the footprint required for integration with an optional liquid sample collection apparatus. For example, the shape, number, and distance between individual posts or columns may be chosen to direct the flow path (e.g., distance between posts in one direction may be smaller than the distance between posts in the other direction to create choke-points) and increase capillary force (e.g., higher density, greater force), while still holding the necessary volume. For example, the capillary bed design of the first lyophilized chamber module 120 may have a large surface area due at least in part to the incorporation of a high density of the posts or columns, which may be tailored to encourage even and repeatable filling under conditions that may be seen in the field (e.g. rocking, jostling, etc.) as well as aid in cake formation during the lyophilization process. The large surface area along with the plurality of posts or columns encourages even and repeatable filing. For example, reliable filling in the field may be realized when the structure of the first lyophilized chamber module 120 exerts a higher capillary force on the filling fluid (e.g., liquid sample being prepared before subsequent DNA sequencing) than the other forces acting on the fluid including the gravitational force.

In certain example embodiments, the first lyophilized chamber module 120 comprises a microfluidic chamber having an average depth from about 5 to about 2500 microns, such as at least about any of the following: 5, 10, 25, 50, 80, 100, 120, 150, 180, and 200 microns, and/or at most about any of the following: 2500, 2000, 1500, 1000, 800, 750, 700, 650, 600, 550, 500, 450, 400, 350, 300, 250, and 200 microns.

As noted above, a first mixing module 130 is incorporated between the first lyophilized chamber module 120 and PCR module 140 to ensure even distribution of reagents to optimize performance in the PCR module 140. In accordance with certain example embodiments and as illustrated in FIG. 1, the first mixing module 130 may comprise a plurality of separate first mixing inlet channels 134 operatively connected to the first mixing module inlet, in which the liquid sample is separated across each of the plurality of separate first mixing inlet channels 134 and pooled in the one or more first mixing pools 132. The first mixing module 130 may also comprise a plurality of separate first mixing outlet channels 136 operatively connected to the first mixing module outlet. In this regard, the liquid sample after reconstituting the lyophilized reagents from the first lyophilized chamber module 120 may be split into a plurality of smaller streams, pooled in one or more separated first pools, and recombined into a single stream prior to entering the PCR module 140. Although the first mixing module 130 is illustrated in FIG. 1 as a pooling mixer, certain example embodiments may not utilize such a structure for the first mixing module 130. For example, the first mixing module 130 may comprise a static mixer or a plurality of micro-fluidic channels in which the lyophilized constituents are admixed into the liquid sample.

In certain example embodiments, the one or more first mixing pools 132 may have an average depth from about 50 to about 600 microns, such as at least about any of the following: 50, 100, 150, 200, 220, 250, 280, 300, 320, and 350 microns, and/or at most about any of the following: 600, 580, 550, 520, 500, 480, 460, 440, 420, 400, 380, and 350 microns.

After exiting the first mixing module 130, the liquid sample (now including the reconstituted primers and PCR master mix) enters the PCR module 140 for amplification. In accordance with certain example embodiments, the PCR module 140 may utilize a micro-fluidic PCR approach where temperature cycling can be achieved by continuously flowing the reaction mixture (i.e., the liquid sample including the reconstituted primers and PCR master mix) through different temperature zones in the serpentine path. The temperatures necessary to cycle through denaturation, annealing, and extension steps may be controlled via integrated heaters (e.g., strip heaters along with thermocouples or other temperature measurement devices that provide feedback of localized temperatures), and heating/cooling rates may be controlled by varying the cross-sectional area of the micro-fluidic channels, thickness of the base substrate, and/or reaction mixture flow rate. A software package may monitor and control multiple separate temperature zones. For example, software may monitor and control three separate temperature zones using the integrated heaters to provide zones of denaturing, annealing and extension steps. Modification of temperature set-points for separate temperature zones (e.g., three temperature zones for denaturing, annealing, and/or extension) alone or in combination with modifications to the geometry of the serpentine path allow this device to be optimized for a variety of PCR reactions. Such modifications, as briefly noted above, may include temperature changes, ramp rates, reaction times, and liquid volumes. In certain example embodiments, the surface of the PCR module 140 may be, for example, functionalized or otherwise treated to reduce non-specific protein binding and/or modify the hydrophobicity of the substrate. In this regard, for example, certain example embodiments include one or more (e.g., all) of the modules functionalized as noted above, for example, to prevent bio-fouling of the micro-fluidic chip surface. Additionally or alternatively, treatment of the PCR module 140 to reduce non-specific protein binding may include, for example, surface polishing to smooth the serpentine channel.

In certain example embodiments, the PCR module 140 may utilize ramp rates from hot to cold and vice versa that may be identical. In certain example embodiments, the PCR module 140 may utilize a gradual heating and rapid cooling of the liquid sample during continuous flow through the serpentine micro-fluidic channel. By way of example only, the liquid sample may be steadily heated through the serpentine micro-fluidic channel at a constant rate between about 1 and 4° C./s for sample extension and denaturing then rapidly cooled at a rate above 10°/s. In certain other example embodiments, the heating ramp rates may comprise about 1.75° C./s. These example heating and cooling rates can be controlled in a small form factor by, for example, varying the cross-sectional area of the serpentine micro-fluidic channel and/or the flow rate, and thus localized flow velocity, through an isothermal gradient. Such temperature rates, by way of example only, may be realized by resistive microstrip heaters held at 95° C. and 60° C. As noted above, the plurality of discrete heaters may be in operative communication, such a via a common bus as noted above, with a plurality of predetermined zones of the serpentine micro-fluidic channel and define denaturing zones, annealing zones, and extension zones along a length of the serpentine microfluidic channel. In this regard, the PCR module 140 may perform multiple cycles (e.g., 10-50 cycles) prior to the liquid sample leaving the PCR module 140. In accordance with certain example embodiments, the PCR module may also include an initial denature (e.g., 5 minutes) and final extension (e.g., 5 minutes) times that act to increase the efficiency of the reaction. By way of example only, the initial denature may be performed in the horizontal channel at the bottom of the PCR module 140 and final extend may be the pattern of smaller channels on the left-hand side of the PCR module 140.

In certain example embodiments, the serpentine micro-fluidic channel of PCR module 140, may comprise a uniform cross-section along a total length of the serpentine micro-fluidic channel. Alternatively, the serpentine micro-fluidic channel may comprise a variable cross-section along a total length of the serpentine micro-fluidic channel, including a first cross-section at a first location and a second cross-section at a second location in which the first cross-section is larger than the second cross-section. Alternatively, the serpentine micro-fluidic channel may have a constant cross-section. For example only, the serpentine micro-fluidic channel may have a constant width of about 250 microns (e.g., +/−about 5, 10, 15, 20, 25, 30, 40, or 50 microns), and a constant depth of about 300 microns (e.g., +/−about 5, 10, 15, 20, 25, 30, 40, or 50 microns). In accordance with certain example embodiments, the depth of the serpentine micro-fluidic channel may be about 10% to about 40% larger than the width of the serpentine micro-fluidic channel, such as at least about any of the following: 10, 15, 20, and 25% larger, and/or at most about any of the following: 40, 35, 30, and 25% larger.

In certain example embodiments, the serpentine micro-fluidic channel may have an average depth from about 200 to about 750 microns, such as at least about any of the following: 200, 220, 250, 280, 300, 320, and 350 microns, and/or at most about any of the following: 750, 700, 650, 600, 580, 550, 520, 500, 480, 460, 440, 420, 400, 380, and 350 microns.

Subsequent to amplification in the PCR module 140, the now amplified liquid sample enters the purification module 150. In this regard, the purification module 150 may extract and purify the amplified DNA present in the liquid sample. As noted above, the purification module 150 may include an active region followed by a valve (or similar control mechanism) that switches flow between a waste outlet and downstream modules in the multi-module sample preparation device (e.g., micro-fluidic chip). The active region, for example, may comprise of either a packed bead bed or periodic array of surface functionalized structures (e.g., pillars, beads, lengthwise columns, etc.) that will specifically bind to and release the amplified DNA regions upon exposure to various reagents and/or stimuli (e.g., temperatures, pH, light, etc.). Similar methods may be employed by which the amplified DNA is captured and purified with a series of wash and elution steps. One example procedure may involve washing the amplified DNA received from the PCR module 140 over a functionalized surface, rinsing the purification module with a wash buffer, and then eluting the amplified DNA to the downstream modules. For instance, one example procedure may involve washing the amplified DNA received from the PCR module 140 over a chitosan functionalized PMMA surface with an acidic solution (i.e. pH <6), rinsing the purification module 150 with an acidic wash buffer to waste, and then eluting the amplified DNA with a basic solution (e.g., pH >8) to the downstream modules. The waste outlet, for example, may shunt excess reagents to waste and then actively switch fluid paths using a pinch valve or similar on the waste outlet. In certain example embodiments, therefore, the purification module 150 may comprise one or more mobile phase inlets in operative communication with the active region. The purification module 150 also comprises a pinch valve or a diverter valve comprising a first orientation that defines the first pathway from the purification outlet to the waste outlet and a second orientation that defines a second pathway from the purification outlet to a purified stream outlet that leads to downstream modules.

As noted above, the solid phase of the purification module 150 may comprise a packing media having a functionalized surface configured to bind and release the one or more amplified target DNA regions. The packing media can include but is not limited to commercial beads optimized for DNA clean up (e.g., AMPure XP beads), standard common materials (e.g., silica beads), or functionalized substrates (e.g., PMMA functionalized with chitosan, hydroxypropylmethyl-cellulose, or poly(vinyl alcohol)).

Subsequent to the purification module 150, the purified liquid sample having the amplified DNA may be reacted with adapter sequences for enabling DNA sequencing of the amplified DNA regions. Accordingly, the next steps in sample processing may include reconstitution of lyophilized sequencing reagents to modify the purified sequencing reagents for sequencing. The adapter sequences and buffer components required for sequencing, such as by a MinION sequencer, may be less stable in liquid form for prolonged periods. As such, these adapter sequences and buffer components may be lyophilized in micro-fluidic lyophilized chamber modules modeled after capillary pumps and as described above. As noted above when discussing the first lyophilized chamber module, these designs enable the liquid reagents to be readily dispersed over a large surface area and dried down in a lyophilization chamber to form a lyophilized chamber. The large surface area also aids in quick reconstitution of reagents; however, such a design if too deep, is highly susceptible to inconsistent fluid flow and bubble entrapment when not operated on a level plane. Therefore, the lyophilized chambers may have a much shallower depth than the other modules, and features may be spaced-out such that fluid follows a general path in the lyophilized chambers, while still allowing for mixing therein. For example, the characteristic dimension (i.e., depth for the lyophilized chambers) has the greatest effect on capillary force and may in certain example embodiments be minimized to ensure a reliable filling pattern.

In certain example embodiments, the reagents in the lyophilized chambers modules (i.e., the second lyophilized chamber module 160 and the third lyophilized chamber module 170) can be reconstituted in parallel. For instance, reagents in the third lyophilization chamber module 170 may be reconstituted with a reconstituting fluid (e.g., water) while reagents in the second lyophilized chamber module 160 may be reconstituted with the purified DNA solution and then incubated in a long channel 162 (e.g., a delay circuit) as shown in FIG. 1, allowing the adapter sequences to attach to the amplified DNA. After this incubation the adapted DNA sample may flow into a second mixing module 180 followed in series with the reconstituted sequencing buffer from a third lyophilized chamber module 170 as shown in FIG. 1. The sequential addition of reagents to a mixing module is a unique problem in micro-fluidics, where mixers are generally designed to have to simultaneous inputs. Therefore, the second mixing module 180 in accordance with certain example embodiments first splits the first solution (e.g., the adapted DNA sample exiting the delay circuit) into several parallel chambers, designed as large open areas that may contain a plurality of posts, which force mixing with the reagents that follow (i.e., from the third lyophilized chamber module 170). Finally, the mixed fluid in the parallel chambers are recombined to ensure all components flow back together before moving onto a DNA sequencer and/or a sequencer interface.

As noted above, the purified liquid sample leaving the purification module 150 may be directed to or passed into the second lyophilized chamber module 160. Similar to the first lyophilized chamber module 120, the second lyophilized chamber module 160 may comprise a capillary bed configuration that exerts a capillary force on the liquid sample that is greater than gravitational forces acting on the liquid sample. For example, the capillary bed configuration may include a plurality of posts or columns that may extend at least a portion (or completely) of the distance of the bed depth, in which the plurality of posts or columns may be provided in a high density. In this regard, the bed depth and the distance between the posts or columns in both the X and Y directions impact the filling properties and strength of the capillary force. In accordance with certain example embodiments, the capillary bed configuration may be formed with at least the following three (3) design criteria: (1) hold the correct volume; (2) maximize capillary force; and (3) fit within the footprint required for integration with an optional liquid sample collection apparatus. For example, the shape, number, and distance between individual posts or columns may be chosen to direct the flow path (e.g., distance between posts in one direction may be smaller than the distance between posts in the other direction to create choke-points) and increase capillary force (e.g., higher density, greater force), while still holding the necessary volume. For example, the capillary bed design of the first lyophilized chamber module 120 may have a large surface area due at least in part to the incorporation of a high density of the posts or columns, which may be tailored to encourage even and repeatable filling under conditions that may be seen in the field (e.g. rocking, jostling, etc.) as well as aid in cake formation during the lyophilization process. The large surface area along with the plurality of posts or columns encourages even and repeatable filing. For example, reliable filling in the field may be realized when the structure of the first lyophilized chamber module 120 exerts a higher capillary force on the filling fluid (e.g., liquid sample being prepared before subsequent DNA sequencing) than the other forces acting on the fluid including the gravitational force.

In certain example embodiments, the second lyophilized chamber module 160 comprises a microfluidic chamber having an average depth from about 5 to 2500 microns, such as at least about any of the following: 5, 10, 25, 50, 80, 100, 120, 150, 180, and 200 microns, and/or at most about any of the following: 2500, 2000, 1500, 1000, 800, 750, 700, 650, 600, 550, 500, 450, 400, 350, 300, 250, and 200 microns.

In certain example embodiments and as shown in FIG. 1, the second lyophilized chamber module 160 comprises a delay circuit 162 configured to provide a desired residence time for attachment of the adapter sequences to the one or more amplified target DNA regions. For example, the length and/or cross-section of the delay circuit 162 (e.g., winding micro-channel) may be configured to ensure a residence time therethrough from about 2 to about 10 minutes, such as at least about any of the following: 2, 3, 4, and 5 minutes, and/or at most about any of the following: 10, 9, 8, 7, 6, and 5 minutes.

As noted above and as illustrated in FIG. 1, the multi-module sample preparation device 100 includes a second mixing module 180 downstream from both the second lyophilized chamber 160 and the third lyophilized chamber 170. In certain example embodiments, the one or more second mixing pools 182 of the second mixing module 180 are located between and operatively connected to a plurality of separate second mixing inlet channels 184 operatively connected to the second mixing module inlet and a plurality of separate second mixing outlet channels 186 operatively connected to the sample outlet 105.

In certain example embodiments, the one or more second mixing pools 180 may have an average depth from about 50 to about 600 microns, such as at least about any of the following: 50, 100, 150, 200, 220, 250, 280, 300, 320, and 350 microns, and/or at most about any of the following: 600, 580, 550, 520, 500, 480, 460, 440, 420, 400, 380, and 350 microns.

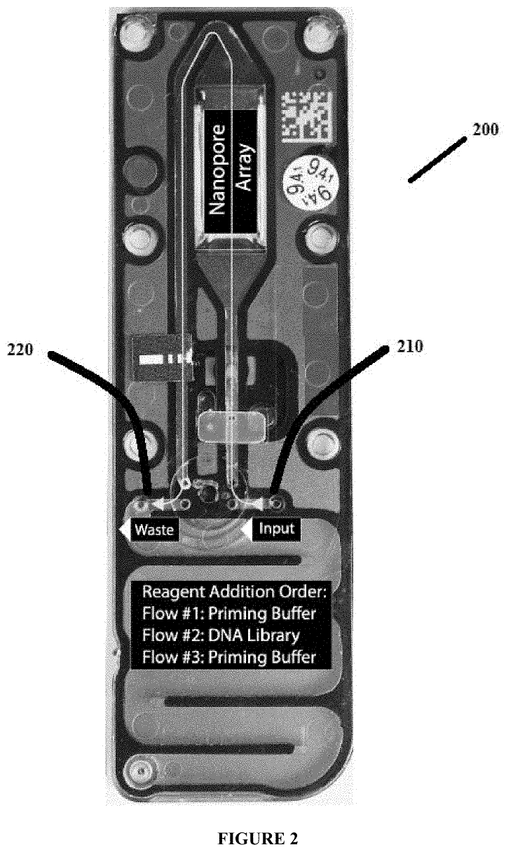

In accordance with certain example embodiments, the multi-module sample preparation device 100 may further comprise and/or be operatively connected to a sequencer interface module, such as via the sample outlet 105. The sequencer interface module, for instance, may be operatively connected to the sample outlet 105 of the multi-module sample preparation device 100 and a DNA sequencer. Although the DNA sequencer may not be particularly limited, FIG. 2 shows a schematic of one example DNA sequencer (e.g., flow cell) 200 including a sequencer inlet 210 (e.g., port) and a sequencer outlet 220 (e.g., port). Operation of the DNA sequencer may comprise flushing or priming the flow cell of the DNA sequencer with a priming buffer followed by flowing a sequencing-ready liquid sample (e.g., sample leaving the multi-module sample preparation device 100) through the flow cell, followed by clearing the flow cell with another volume of priming or storage buffer. The sequencer interface module, for example, may comprise at least one interface outlet configured to deliver a sample (e.g., a sample exiting the multi-module sample preparation device 100 via the sample outlet 105) for sequencing.

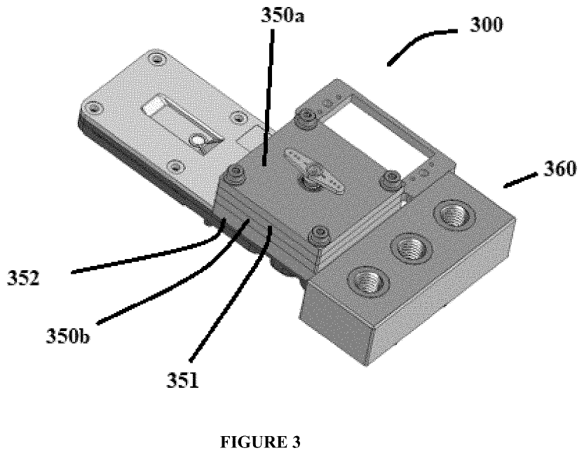

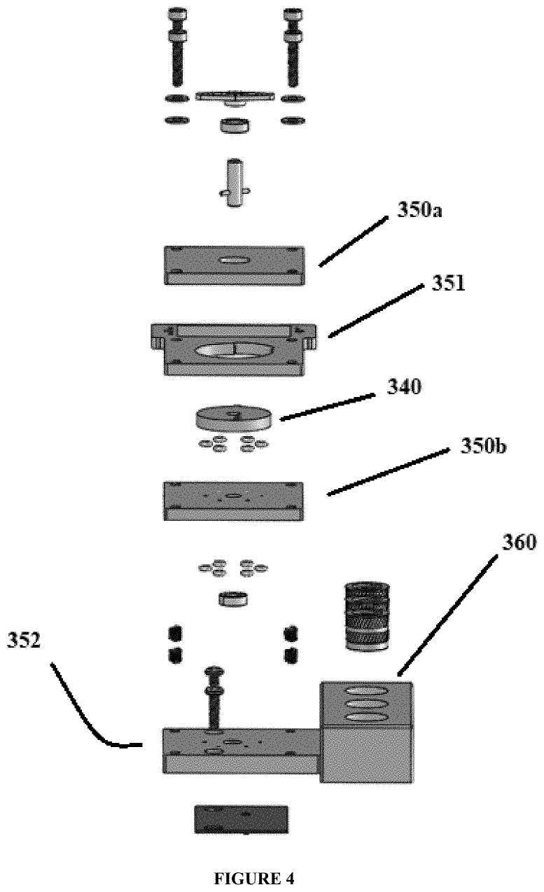

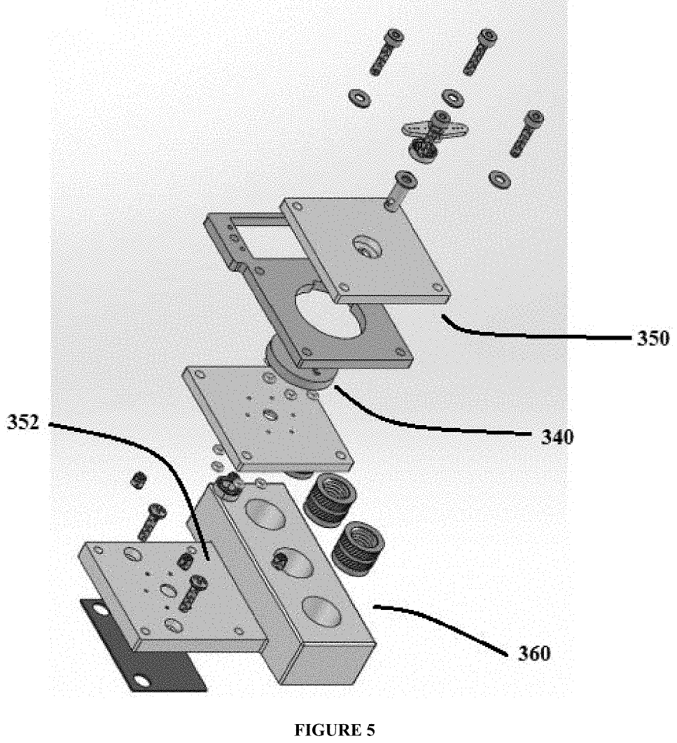

FIG. 3 illustrates a sequencer interface 300 in an assembled configuration according to certain example embodiments. As shown in FIG. 3, the sequencer interface may comprise a manifold component including a bottom mounting component 352 as well as an inlet and outlet manifold 360, in which these component may be formed as separate pieces and assembled together or formed as a single unitary structure. As also shown in FIG. 3, the sequencer interface may include rotary valve component including a multi-port rotary valve 340 (shown in FIGS. 4 and 5) housed or incased within an orifice of a housing component 351. The rotary valve component may also include a top cover plate 350a and bottom cover plate 350b that sandwich the housing component 351 and the multi-port rotary valve 340, which is rotatably housed within the housing component 351 in accordance with certain example embodiments.

FIGS. 4 and 5 illustrate alternative exploded views of a sequence interface 300 including the multi-port rotary valve 340 that will be mounted between a bottom mounting component 352 while the inlet and outlet manifold 360 is provided adjacent the bottom mounting component (either as separate assembled pieces or as a unitary structure). In accordance with certain example embodiments, for example, the bottom mounting component 352 and the inlet and outlet manifold 360 may be a single or unitary structure (e.g., formed by 3D printing or other additive manufacturing method, molding, etc.). The multi-port rotary valve 340, as illustrated in FIG. 4, is housed or incased with an orifice of a housing component 351. Top cover plate 350a and bottom cover plate 350b sandwich the multi-port rotary valve 340 that is rotatably housed within the housing component 351 in accordance with certain example embodiments.

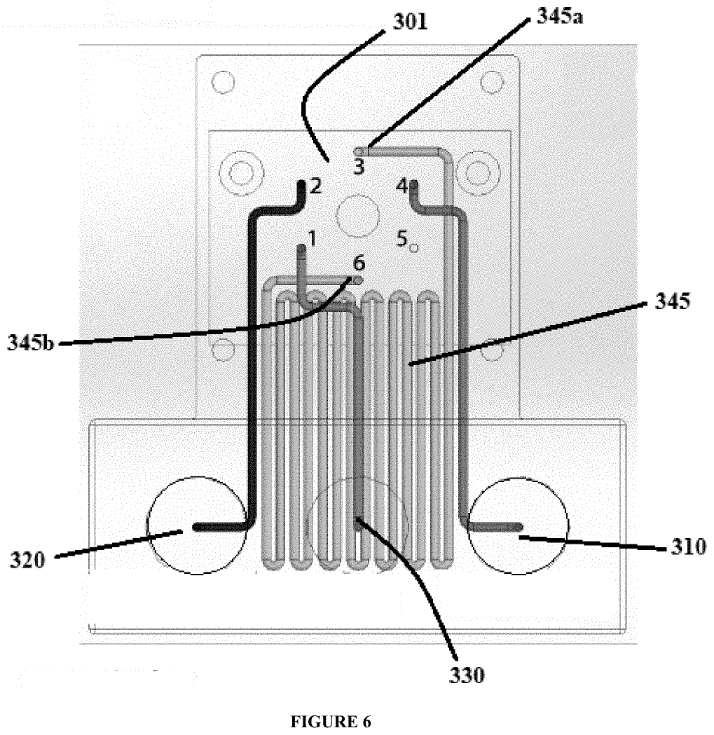

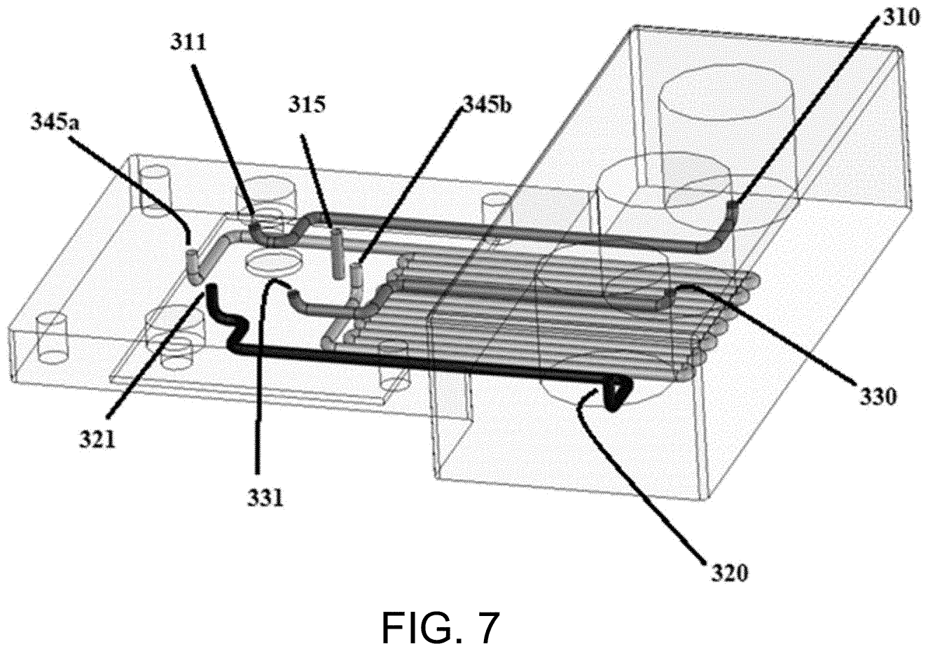

The sequencer interface module 300, as illustrated by FIG. 3 and noted above, may comprise a manifold component having, for example, six ports 301 (i.e., port numbers 1 through 6) aligned in circle as shown in FIG. 6, which will be aligned with the multi-port rotary valve 340 as discussed in greater detail below. In this regard, the manifold component may include a priming buffer inlet 310, a sequencing-ready liquid sample inlet 320, and a waste outlet 330. FIG. 7 illustrates a transparent schematic of the manifold component of FIG. 6 that illustrates the respective orientation of the ports (i.e., port number 1-6 in FIG. 6) associated with the manifold component of the sequence interface in accordance with certain example embodiments. As shown in FIG. 7, the manifold component includes a pathway 315 that interfaces with a DNA sequencer and an injection loop 345 having a first end 345a and a second end 345b. The priming buffer inlet 310 is associated with a primer buffer pathway 311. The waste outlet 330 is associated with waste pathway 331, and the sequencing-ready liquid sample inlet 320 is associated with a sample pathway 321.

In certain example embodiments, the priming buffer inlet 310, the sequencing-ready liquid sample inlet 320, and the waste outlet 330 are each operatively connected to a multi-port rotary valve 340 (best shown in FIGS. 4, 5, 8, and 9) having a first valve position configured to (i) operatively connect the priming buffer inlet 310 to an inlet of a DNA sequencer (i.e., illustrated as port 5 on FIG. 6 and pathway 311 in FIG. 7), (ii) operatively connect the sequencing-ready liquid sample inlet 320 to a first end 345a of an injection loop 345, and (iii) operatively connect the waste outlet 330 to a second end 345b of the injection loop 345. In certain example embodiments, the multi-port rotary valve 340 has a second valve position configured to (i) operatively connect the second end 345b of the injection loop 345 to the DNA sequencer via pathway 311 as shown on FIG. 7 and port number 5 on FIG. 6, (ii) operatively connect the priming buffer inlet 310 to the first end 345a of the injection loop 345, and (iii) operatively connect the sequencing-ready liquid sample inlet 320 to the waste outlet 330.

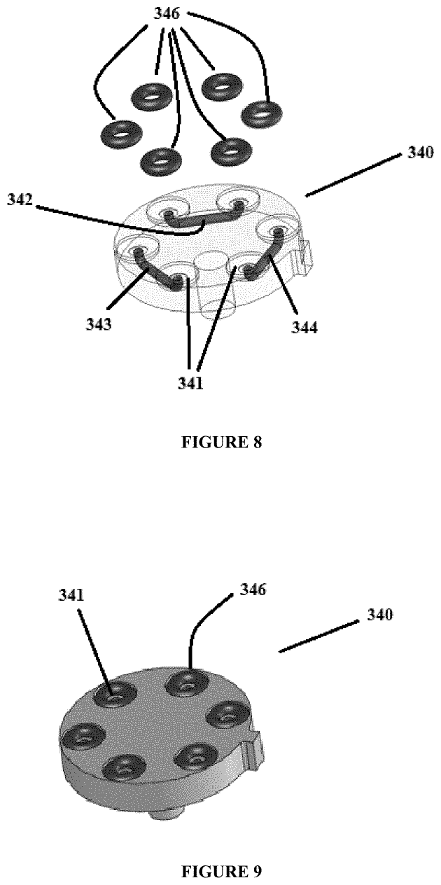

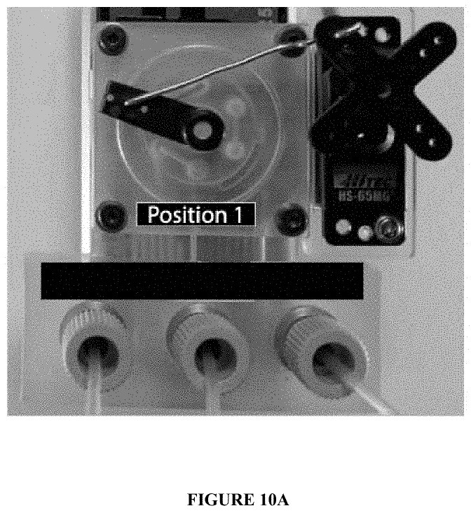

In certain example embodiments and as illustrated in FIGS. 8 and 9, the multi-port rotary valve 340 includes a plurality of ports 341 that align with port numbers 1-6 as shown in FIGS. 6 and 7 to complete fluid pathways through the manifold component and the multi-port rotary valve. Although not shown in the figures, the multi-port rotary valve and/or the manifold component may be configured to include face seals that define or complete the fluid pathways. In this regard, the completed fluid pathways may not extend into and through the body of the manifold component, but flow through grooves formed in the surface of the manifold component. As shown in FIG. 8, the multi-port rotary valve 340 includes through-channels 342, 343, and 344 formed therein, and being configured (i) to operatively connect the priming buffer inlet 310 with the inlet of the DNA sequencer (e.g., via port number 5 in FIG. 6 and pathway 315 in FIG. 7) while in the first position, and (ii) to operatively connect the sequencing-ready liquid sample inlet 320 to the waste outlet 330 while in the second position. It should be noted, however, that the several alternatives to the through-channels 342, 343, and 344 need not always be enclosed within the rotary valve. For example, face seals and grooves formed in the surface of the rotary valve may be utilized to define the respective channels. One important feature associated with the first position is the fact that the sequencing-ready liquid sample inlet 320 is connected to the injection loop 345, in which the injection loop 345 measures out the fluid, and the excess may go to waste. In this regard, the correct volume of the sequencing-ready liquid is housed in the injection loop 345 and ready for injection into the DNA sequencer in position two. For instance, the sequencing-ready liquid is ejected from the injection loop 345 and into the DNA sequencer in position two. As also illustrated by the particular non-limiting embodiment of FIG. 8, the plurality of ports 341 and through-channels 342, 343, and 344 comprise six ports and three through-channels, in which the six ports include a first pair of ports and a first through-channel 342 formed therebetween, a second pair of ports and a second through-channel 343 formed therebetween, and a third pair of ports and a third through-channel 344 formed therebetween.

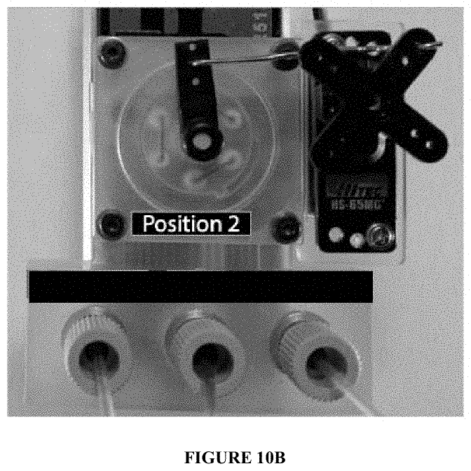

FIG. 10A illustrates the multi-port rotary valve in a first position (e.g., sequencing-ready liquid sample fills an injection loop 345 to measure out the correct volume for subsequent injection into the DNA sequencer) in accordance with certain example embodiments, and FIG. 10B illustrates the multi-port rotary valve in a second position (e.g., priming buffer solution pushes the sequencing-ready liquid sample out of the injection loop 345 and into the DNA sequencer) in accordance with certain example embodiments. In accordance with certain example embodiments, a drive mechanism may be operatively connected to the multi-port rotary valve, and being configured to cycle the multi-port rotary valve between the first position and the second position.

In accordance with certain example embodiments, one or more of the lyophilized chambers may instead include a blister system instead of lyophilized reagents. For example, the blister system may comprise self-contained fluid reservoirs that can be punctured or otherwise broken open to release their contents. By way of example only, the first lyophilized chamber may include a first plurality of blisters including the reagents associated with the first lyophilized chamber, the second lyophilized chamber may include a second plurality of blisters including the reagents associated with the second lyophilized chamber, and/or the third lyophilized chamber may include a third plurality of blisters including the reagents associated with the third lyophilized chamber. Blister systems, in accordance with certain example embodiments, may also be used for the reconstitution fluid and/or for the purification reagents.

In accordance with certain example embodiments, the multi-module sample preparation device (e.g., micro-fluidic chip) may include an integrated bubble trap. For example, the bubble trap may be incorporated into the microfluidic manifold. Bubble traps, for example, may comprise microporous membranes that will allow air to pass out of the device, but not fluids. By way of example only, the microporous membrane may comprise polytetrafluoroethylene (PTFE). The bubble trap, for example, may be integrated into the manifold of the sequencer interface as the last step before feeding the sample into the DNA sequencer. In certain example embodiments, the bubble trap may be located between the end of the injection loop 345b and injection port 315. In this regard, the bubble trap may protect the DNA sequencer from air bubbles hat may damage, for example, a nanopore array in the DNA sequencer.

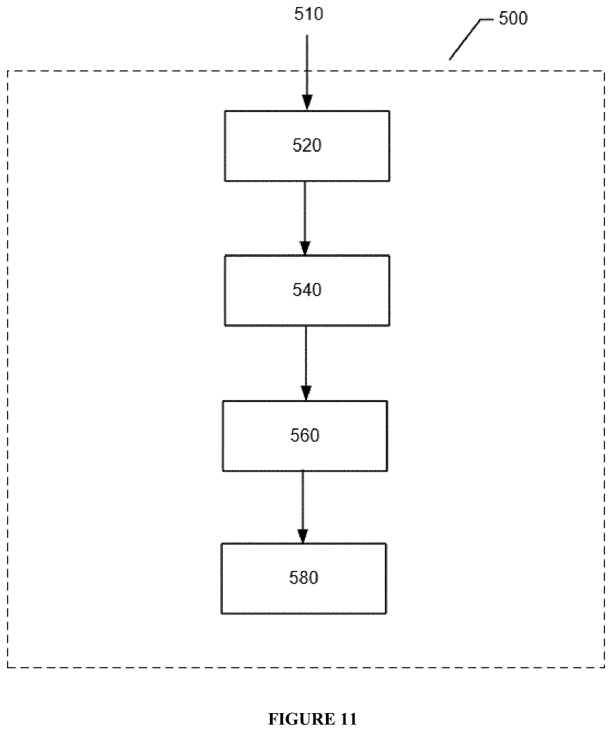

In another aspect and as illustrated by FIG. 11, the invention provides a system 500 including an optional liquid sample collection apparatus 520, which is operatively connected to a system inlet 510 for the collection of one or more liquid samples (e.g., water from a natural source), including a collection apparatus outlet and a multi-module sample preparation device 540, such as those described and disclosed herein, in which the sample inlet of the multi-module sample preparation device is in operative communication with the collection apparatus outlet. The system 500 may also comprise a DNA sequencer 580, in which the DNA sequencer is in operative communication (e.g., via a sequencer interface as described and disclosed herein) with the sample outlet of the multi-module sample preparation device. In certain example embodiments, the system may include a sequencer interface module 560 located between and operative communication with the sample outlet of the multi-module sample preparation device 540 and the DNA sequencer 580. The system according to certain example embodiments may be embodied within, for example, a submersible autonomous underwater vehicle (AUV) that may collect water samples from natural and/or manmade bodies of water for eDNA analysis. In accordance with certain example embodiments, the liquid sample collection apparatus (if present) may collect one or more liquid samples and perform a lysis process on the one or more samples to provide lysate for sample preparation by the multi-module sample preparation device 540.

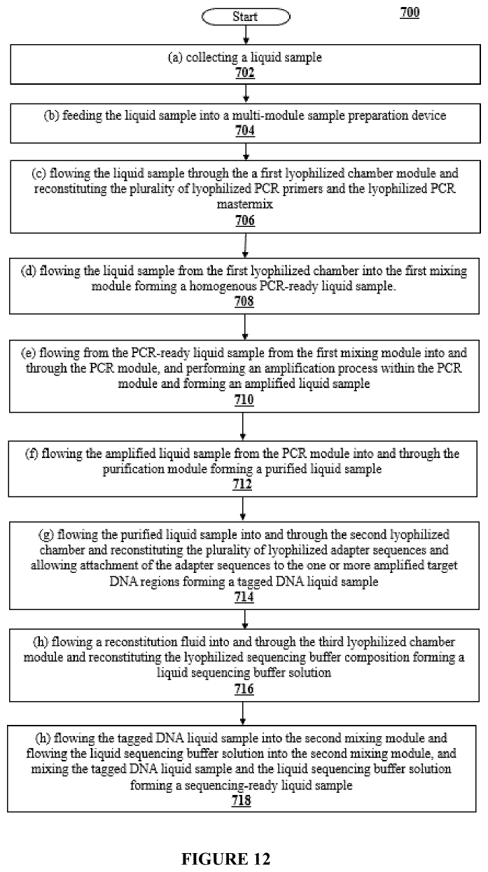

In yet another aspect and as illustrated by FIG. 12, the present invention provides a method 700 of preparing a sample for DNA sequencing, in which the method may include the following: (a) optionally collecting a liquid sample 702, (b) feeding the liquid sample (e.g., a lysed liquid sample) into a multi-module sample preparation device 704, such as those described and disclosed herein; (c) flowing the liquid sample through a first lyophilized chamber module and reconstituting the plurality of lyophilized PCR primers and the lyophilized PCR master mix 706; (d) flowing the liquid sample from the first lyophilized chamber into the first mixing module forming a homogenous PCR-ready liquid sample 708; (e) flowing the PCR-ready liquid sample from the first mixing module into and through the PCR module, and performing an amplification process within the PCR module and forming an amplified liquid sample 710; (f) flowing the amplified liquid sample from the PCR module into and through the purification module forming a purified liquid sample 712; (g) flowing the purified liquid sample into and through the second lyophilized chamber and reconstituting the plurality of lyophilized adapter sequences and allowing attachment of the adapter sequences to the one or more amplified target DNA regions forming a sequence-able DNA liquid sample 714; (h) flowing a reconstitution fluid into and through the third lyophilized chamber module and reconstituting the lyophilized sequencing buffer composition forming a liquid sequencing buffer solution 716; and (i) flowing the sequence-able DNA liquid sample (e.g., DNA containing adapter sequences) into the second mixing module and flowing the liquid sequencing buffer solution into the second mixing module, and mixing the sequence-able DNA liquid sample and the liquid sequencing buffer solution forming a sequencing-ready liquid sample 718. In accordance with certain example embodiments, the method may comprise performing a lysis process on the liquid sample to produce lysate for feeding into the multi-module sample preparation device.

These and other modifications and variations to embodiments may be practiced by those of ordinary skill in the art without departing from the spirit and scope, which is more particularly set forth in the appended claims. In addition, it should be understood that aspects of the various embodiments may be interchanged in whole or in part. Furthermore, those of ordinary skill in the art will appreciate that the foregoing description is by way of example only, and it is not intended to limit the invention as further described in such appended claims. Therefore, the spirit and scope of the appended claims should not be limited to the exemplary description of the versions contained herein.

That which is claimed:

1. A multi-module sample preparation device, comprising:
(a) a sample inlet for receiving a liquid sample comprising DNA;
(b) a sample outlet;
(c) a waste outlet; and
(d) a plurality of operatively connected modules including:
(i) a first lyophilized chamber module comprising a plurality of lyophilized PCR primers and a lyophilized PCR master mix including one or more deoxynucleoside triphosphates (dNTPs), one or more buffers, and/or one or more polymerases, the first lyophilized chamber module includes a first lyophilized chamber inlet operatively connected to the sample inlet, and a first lyophilized chamber outlet;
(ii) a first mixing module, the first mixing module includes a first mixing module inlet operatively connected to the first lyophilized chamber outlet, and a first mixing module outlet;
(iii) a PCR module comprising a serpentine microfluidic channel and a plurality of discrete heaters in operative communication with a plurality of predetermined zones of the serpentine microfluidic channel oriented to produce one or more amplified target DNA regions, the PCR module includes a PCR inlet operatively connected to the first mixing module outlet, and a PCR outlet;
(iv) a purification module comprising an active region including a solid phase configured to bind and release the one or more amplified target DNA regions, the purification module includes a purification inlet in operative communication with the PCR module outlet, and a purification outlet being operatively and selectively connected with a first pathway from the purification outlet to the waste outlet and a second pathway from the purification outlet to a purified stream outlet;
(v) a second lyophilized chamber module comprising a plurality of lyophilized adapter sequences for enabling sequencing of the amplified target DNA regions, the second lyophilized chamber module includes a second lyophilized chamber module inlet operatively connected to the purified stream outlet, and a second lyophilized chamber module outlet;
(vi) a third lyophilized chamber module comprising a lyophilized sequencing buffer composition, the third lyophilized chamber module includes a third lyophilized chamber module inlet operatively connected to a source of a reconstitution fluid, and a third lyophilized chamber module outlet; and
(vii) a second mixing module comprising one or more second mixing pools, the second mixing module includes one or more second mixing module inlets operatively connected to the second lyophilized chamber module outlet and the third lyophilized chamber module outlet, and a second mixing module outlet connected to the sample outlet.

2. The multi-module sample preparation device of claim 1, wherein the first lyophilized chamber module comprises a microfluidic chamber having an average depth from about 5 to about 750 microns.

3. The multi-module sample preparation device of claim 1, wherein the first lyophilized chamber module comprises a capillary bed configuration that exerts a capillary force on the liquid sample that is greater than gravitational forces acting on the liquid sample.

4. The multi-module sample preparation device of claim 1, wherein the first mixing module comprises a serpentine, non-pooling mixer.

5. The multi-module sample preparation device of claim 1, wherein the first mixing module comprises one or more first mixing pools having an average depth from about 50 to about 600 microns.

6. The multi-module sample preparation device of claim 1, wherein plurality of discrete heaters in operative communication with a plurality of predetermined zones of the serpentine microfluidic channel defines denaturing zones, annealing zones, and extension zones along a length of the serpentine microfluidic channel.

7. The multi-module sample preparation device of claim 1, wherein the serpentine microfluidic channel comprises a uniform cross-section along a length of the serpentine microfluidic channel.

8. The multi-module sample preparation device of claim 1, wherein the purification module comprises one or more mobile phase inlets in operative communication with the active region, the purification module also comprises a valve comprising a first orientation that defines the first pathway from the purification outlet to the waste outlet and a second orientation that defines the a second pathway from the purification outlet to a purified stream outlet.

9. The multi-module sample preparation device of claim 1, wherein the solid phase of the purification module comprises packing media or a functionalized surface configured to bind and release the one or more amplified target DNA regions.

10. The multi-module sample preparation device of claim 1, wherein the second lyophilized chamber module comprises a microfluidic chamber having an average depth from about 25 to about 750 microns.

11. The multi-module sample preparation device of claim 1, wherein the second lyophilized chamber module comprises a capillary bed configuration that exerts a capillary force on the liquid sample that is greater than gravitational forces acting on the liquid sample.

12. The multi-module sample preparation device of claim 1, wherein the second lyophilized chamber module comprises a delay circuit configured to provide a desired residence time for attachment of the adapter sequences to the one or more amplified target DNA regions.

13. The multi-module sample preparation device of claim 1, wherein the one or more second mixing pools of the second mixing module located between and operatively connected to a plurality of separate second mixing inlet channels operatively connected to the second mixing module inlet and a plurality of separate second mixing outlet channels operatively connected to the sample outlet.

14. The multi-module sample preparation device of claim 1, further comprising a sequencer interface module operatively connected to the sample outlet, the sequencer interface comprising at least one interface outlet configured to deliver a sample for sequencing.

15. The multi-module sample preparation device of claim 14, wherein the sequencer interface module comprises a priming buffer inlet, a sequencing-ready liquid sample inlet, and a waste outlet, and wherein the priming buffer inlet, the sequencing-ready liquid sample inlet, and the waste outlet are each operatively connected to a multi-port rotary valve having a first valve position configured to (i) operatively connect the priming buffer inlet to an inlet of a DNA sequencer, (ii) operatively connect the sequencing-ready liquid sample inlet to a first end of an injection loop, and (iii) operatively connect the waste outlet to a second end of the injection loop, and wherein the multi-port rotary valve has a second valve position configured to (i) operatively connect the second end of the injection loop to the DNA sequencer, (ii) operatively connect the priming buffer inlet to the first end of the injection loop, and (iii) operatively connect the sequencing-ready liquid sample inlet to the waste outlet.

16. The multi-module sample preparation device of claim 15, wherein the multi-port rotary valve includes a plurality of ports and fluid pathways formed therein, and being configured (i) to operatively connect the priming buffer inlet with the inlet of the DNA sequencer while in the first position, and (ii) to operatively connect the sequencing-ready liquid sample inlet to the waste outlet.

17. The multi-module sample preparation device of claim 16, wherein the plurality of ports and through-channels comprise six ports and three through-channels, wherein the six ports include a first pair of ports and a first through-channel formed therebetween, a second pair of ports and a second through-channel formed therebetween, and a third pair of ports and a third through-channel formed therebetween.

18. The multi-module sample preparation device of claim 15, further comprising a drive mechanism operatively connected to the multi-port rotary valve, and being configured to cycle the multi-port rotary valve between the first position and the second position.

19. A system, comprising:
(a) an optional liquid sample collection apparatus including a collection apparatus outlet;
(b) a multi-module sample preparation device according to claim 1, wherein the sample inlet of the multi-module sample preparation device is in operative communication with the collection apparatus outlet; and
(c) a sequencer, wherein the sequencer is in operative communication with the sample outlet of the multi-module sample preparation device.

20. A method of preparing a sample for DNA sequencing, comprising:
(a) collecting a liquid sample,
(b) feeding the liquid sample into a multi-module sample preparation device according to claim 1,
(c) flowing the liquid sample through the a first lyophilized chamber module and reconstituting the plurality of lyophilized PCR primers and the lyophilized PCR mastermix;
(d) flowing the liquid sample from the first lyophilized chamber into the first mixing module forming a homogenous PCR-ready liquid sample;
(e) flowing from the PCR-ready liquid sample from the first mixing module into and through the PCR module, and performing an amplification process within the PCR module and forming an amplified liquid sample;
(f) flowing the amplified liquid sample from the PCR module into and through the purification module forming a purified liquid sample;
(g) flowing the purified liquid sample into and through the second lyophilized chamber and reconstituting the plurality of lyophilized adapter sequences and allowing attachment of the adapter sequences to the one or more amplified target DNA regions forming a sequence-able DNA liquid sample;
(h) flowing a reconstitution fluid into and through the third lyophilized chamber module and reconstituting the lyophilized sequencing buffer composition forming a liquid sequencing buffer solution; and
(i) flowing the sequence-able DNA liquid sample into the second mixing module and flowing the liquid sequencing buffer solution into the second mixing module, and mixing the sequence-able DNA liquid sample and the liquid sequencing buffer solution forming a sequencing-ready liquid sample.
